(19) United States
(12) Patent Application Publication
(10) Pub. No.: US 2022/0268765 A1
(43) Pub. Date: Aug. 25, 2022

(54) METHODS AND KITS FOR UNIVERSAL CALIBRATION OF LATERAL FLOW TESTING

(71) Applicant: Waters Technologies Corporation, Milford, MA (US)

(72) Inventors: Lingyun Chen, Westborough, MA (US); Justine Yu, Chelsea, MA (US)

(73) Assignee: Waters Technologies Corporation, Milford, MA (US)

(21) Appl. No.: 17/673,482

(22) Filed: Feb. 16, 2022

Related U.S. Application Data
(60) Provisional application No. 63/152,087, filed on Feb. 22, 2021.

(51) Int. Cl. G01N 33/53; G01N 33/543; G01N 33/02

(52) U.S. Cl. CPC G01N 33/5308; G01N 33/54388; G01N 33/02

(57) ABSTRACT

The present disclosure provides methods and kits for detecting an analyte within a sample by using lateral flow testing. Specifically, the present disclosure relates to use of an analyte specific calibration curve for determining quantity of an analyte within a complex sample, wherein the sample is purified/enriched by using a sample preparation method including an affinity resin. The present disclosure further relates to kits including an optional affinity resin, a lateral flow test, and an algorithm corresponding to the analyte specific calibration curve.

### RELATED APPLICATIONS

This application claims the benefit of and priority to U.S. Provisional Patent Application No. 63/152,087 filed on Feb. 22, 2021 titled “METHODS AND KITS FOR UNIVERSAL CALIBRATION OF LATERAL FLOW TESTING,” the contents of which are incorporated herein by reference in their entirety.

### FIELD OF THE TECHNOLOGY

The present disclosure relates generally to methods and kits for lateral flow testing. In particular, the present disclosure relates to calibration methods for use with lateral flow testing. The present disclosure further relates to methods to reduce matrix effects on calibration curves of lateral flow tests. Specifically, affinity purification/enrichment sample preparation methods for quantitative lateral flow tests are disclosed herein.

### BACKGROUND

Lateral flow tests are a form of immunoassay in which the test sample flows along a solid substrate via capillary action. They are easy-to-use devices for the detection and quantification of analytes in complex samples, where the sample is placed on a test device and the results are displayed within minutes. Lateral flow tests have achieved considerable commercial success as a valuable tool in medical, veterinary, food, agricultural and environmental settings due to their low development costs and ease of production. The robustness of lateral flow testing devices allows for on-site testing of products at the source, such as at farms or food-preparation facilities.

### SUMMARY

The results of lateral flow tests are mostly qualitative (on/off) or semi-quantitative because the presence of interfering substances within sample matrix presents enormous challenges to accurate detection of analytes in complex samples. Matrix effects can dramatically influence analysis performance for both identification and quantification of an analyte.

Since the matrix has a pronounced effect on calibration of lateral flow tests, when the matrix is changed, a new specific calibration curve needs to be developed for quantitative analysis of the analyte. For example, in order to use the same calibration curve for analysis of the same analyte in different samples, the matrix of each sample should be matched in terms of their composition. Samples with unknown compositions such as e.g. feed formula consisting a random mixture of grains and additives varying from lot to lot, lacks a representative matrix for calibration. Therefore, each sample requires a new specific calibration curve to be used for quantitative lateral flow test. The complexity of generating and employing numerous calibrations to cope with various matrix effect is a limiting factor for quantitative application of a lateral flow test. Although matrix effects cannot be completely avoided, it can be minimized by optimizing sample preparation procedures for lateral flow test.

It is an object of the present disclosure to obviate or mitigate at least one disadvantage of previous devices and methods for quantitative and qualitative detection of the analyte of interest in various samples selected from agricultural sample, an environmental sample, or a biological sample.

In one aspect, the present disclosure relates to methods of determining the concentration of an analyte in a sample using lateral flow test. The method includes eliminating/minimizing matrix effects on results of a lateral flow assay.

In another aspect, the present disclosure provides affinity purification/enrichment sample preparation methods for eliminating/minimizing matrix effects on results of a lateral flow assay. In some embodiments, sample preparation methods further comprise extraction step.

In another aspect, the present disclosure further relates to methods to reduce matrix effects on calibration curves of lateral flow tests.

In one aspect, provided herein is a method of generating an analyte-specific calibration curve for a lateral flow test of an analyte originally contained within any matrix, containing the steps of: providing an analyte standard, wherein quantity of an analyte within the analyte standard is known; serially diluting the analyte standard with an elution solution and a lateral flow diluent mixture to obtain a series of diluted analyte standards, wherein volume of the elution solution and the lateral flow diluent mixture is known; and applying the series of diluted analyte standards to a lateral flow test to obtain the analyte-specific calibration curve.

The method for generating analyte-specific calibration curve for use in lateral flow testing provided herein is different from the conventional methods for obtaining a calibration curve for use in lateral flow testing. The present technology requires dilution of an analyte standard not only with a lateral flow diluent but also with an elution solution. This difference reflects well the idea of using the analyte-specific calibration curve provided in the present application for lateral flow testing of an analyte originally contained within any sample or any matrix, wherein the sample is purified by using a sample preparation method including an elution solution.

In another aspect, provided herein is a method of detecting quantity of an analyte within a sample, wherein quantity of the analyte is unknown; containing the steps of: extracting the analyte with an extraction solution to obtain a sample extract; loading the sample extract in a column, wherein the column comprises an affinity resin; washing off the column by passing a wash solution through the column; eluting the analyte from the column with an elution solution, wherein volume of the elution solution is known; collecting an eluted analyte; diluting the eluted analyte with a lateral flow diluent to obtain a diluted analyte, wherein volume of the lateral flow diluent is known; applying diluted analyte to a lateral flow test; reading the lateral flow test to obtain a detectable signal; and converting the detectable signal from the lateral flow test to a numerical value related to the quantity of the analyte using an analyte-specific calibration curve, wherein the calibration curve correlates the detectable signals to quantity of the analyte.

In one or more embodiments, the kit further comprises sample preparation instructions for removing matrix effects from the sample. In one embodiment, the sample preparation instructions include use of the optional column.

In some embodiments, the analyte-specific calibration curve is capable of detecting quantity of the analyte originally contained within different samples.

In another aspect, provided herein is a kit for detecting quantity of an analyte within a sample, wherein quantity of the analyte is unknown comprising: optionally, a column comprising an affinity resin; a lateral flow test, including a strip for reacting with an analyte; an algorithm corresponding to the analyte-specific calibration curve, wherein the analyte-specific calibration curve is capable of detecting quantity of the analyte within multiple different samples; and instructions for reading the lateral flow test strip and determining quantity of the analyte based on the algorithm corresponding to the analyte-specific calibration curve.

In one or more embodiments, the analyte-specific calibration curve does not change for use in detecting the analyte in different matrix materials.

In one or more embodiments, the analyte-specific calibration curve does not change for use in detecting the analyte originally contained within different samples.

In one or more embodiments, the analyte-specific calibration curve is obtained by using the methods provided herein this present disclosure. In one embodiment, the analyte-specific calibration curve is obtained by any suitable method that provides a calibration curve free from matrix effects.

In some embodiments, the affinity resin comprises affinity ligands selected from analyte specific proteins, antibodies, aptamers, peptides, molecularly imprinted polymers (MIP), or synthetic small-molecular-weight compounds. In a preferred embodiment, the affinity resin comprises an immunoaffinity resin including analyte specific antibodies.

In some embodiments, the sample contains a single analyte or multiple analytes.

In some embodiments, the affinity resin contains multiple affinity ligands to purify multiple analytes at once. In one embodiments, the immunoaffinity resin contains multiple analyte specific antibodies to purify multiple analytes at once.

In some embodiments, the detectable signal from lateral flow test is test to control (T/C) ratios.

In one or more embodiments, the extraction solution comprises organic solvent, water, or a mixture thereof. In one embodiment, the organic solvent is selected from methanol, ethanol, acetonitrile, or a mixture thereof.

In some embodiments, the sample is selected from an agricultural sample, an environmental sample, or a biological sample. In one or more embodiments, the sample is a complex mixture. In one embodiment, the sample is a food source or food product. In another embodiment, the sample is an animal feed or subsequent quantification thereof. In one embodiment, the food source or food product is an animal feed or subsequent quantification thereof.

In one or more embodiments, the analyte is selected from mycotoxins, allergens, antibiotics. In one embodiment, the mycotoxins are selected from the group of aflatoxin, ochratoxin, deoxynivalenol, nivalenol, T2/HT2 toxin, patulin, zearalenone, citrinin, fumonisin or their analogs. In another embodiment, the aflatoxin is selected from the group of aflatoxin B1, aflatoxin B2, aflatoxin G1, aflatoxin G2, aflatoxin M1, or aflatoxin M2.

The present disclosure advantageously provides a universal analyte-specific calibration curve for analysis of different samples by quantitative lateral testing. Extracting the sample with suitable extraction solvent and purifying the sample with an affinity column minimizes the matrix effect; therefore, the need for generating numerous calibration curves for each different sample is eliminated.

The methods and kits provided herein provide time-efficient, cost effective ways for quantitative analysis of analytes of interests with improved sensitivity.

### BRIEF DESCRIPTION OF THE DRAWINGS

The invention will be more fully understood from the following detailed description taken in conjunction with the accompanying drawings, in which:

FIG. 1 illustrates a method of detecting quantity of an analyte within a sample using

an analyte-specific calibration curve for a lateral flow test.

FIG. 2 illustrates a kit for detecting the quantity of an analyte within a sample including optional affinity chromatography column, a lateral flow test, and an analyte-specific curve.

FIG. 3 illustrates T/C ratios for corn and peanut samples spiked with various aflatoxin concentration at ppb level.

FIG. 4 illustrates T/C ratios for three replicates of corn and peanut samples spiked with 10 ppb aflatoxin.

FIG. 5 illustrates overlay of T/C ratios for three replicates of corn and peanut extracts spiked with 10 ppb aflatoxin.

### DETAILED DESCRIPTION

Methods and kits described herein provide methods and kits for quantitatively detecting specific analytes using lateral flow tests without need for generating numerous calibration curves for each sample where analyte is present in.

The present disclosure introduces the concept of using analyte-specific calibration curves for samples which are substantially free of matrix effect. Analyte-specific calibration curves can be used for quantitative determination of a specific analyte regardless of the matrix effect (e.g. matrix of corn vs. matrix of peanut), whereby the need for generating a new calibration curve for each sample matrix is removed.

According to embodiments of the present disclosure, matrix effects can be minimized or eliminated by including an affinity column chromatography sample preparation step within a workflow. Affinity chromatography can be used for purification/enrichment of analytes prior to lateral flow test.

In some examples, the present disclosure includes a method of separating a sample with an analyte of interest. The method includes introducing the sample with the analyte into a chromatographic system. The chromatographic system can include a flow path defined by the interior of the chromatographic system and at least a portion of the interior of the chromatographic system with an affinity resin, such as an immunoaffinity resin. The method further includes eluting the analyte through the flow path, and collecting the eluted sample comprising the analyte. The method can optionally include washing the column after introducing the sample but before eluting analytes(s) from the resin in the column.

In some examples, the present disclosure includes a method of separating a sample with an analyte. The method includes introducing the sample with the analyte to a fluidic system including a flow path defined by an interior of the fluidic system. At least a portion of the interior of the fluidic system can include an affinity resin (e.g., immunoaffinity resin). The method can further include eluting the sample through the fluidic system and collecting the eluted sample comprising the analyte.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the term “matrix” refers to the components of a sample other than the analyte of interest. The matrix can have a considerable effect on the way the analysis is conducted and the quality of the results are obtained.

As used herein, the term “matrix effect” is referred to combined effect of all components of the sample other than the analyte. Components originating from the sample matrix that co-elute with the analytes can interfere with the analyte measurements causing false negative or false positive results.

As used herein, the term “substantially” refers to a quantitative state that indicates a complete or near complete degree or degree of a feature or characteristic of interest. As used herein, the term “substantially free” means that the analyte, the sample, solution, media, supplement, excipient and the like, is at least 85%, at least 90%, at least 95%, at least 98%, or at least 98.5%, or at least 99%, or at least 99.5%, or at least 100% free of interference compounds, impurities, contaminants or equivalent thereof.

As used herein, the term “quantify”, “quantification”, “measure” as used interchangeably, refers to the determination of the amount or concentration of an analyte in a sample, using the method according to the present disclosure. The term “analyte,” as used herein, refers to any molecule of interest that is desirable to quantify using the methods described herein.

As used herein, the term “sample” refers to any medium that includes an analyte (e.g. a mycotoxin) to be quantified using the methods according to the present disclosure. A sample may be selected from an agricultural sample, an environmental sample, or a biological sample. A sample may include, but is not limited to, for example, a food substance (e.g., poultry, fresh meat, milk, yogurt, dairy products, bakery products, beverages, juices, cheeses, vegetables, fruit, fish, etc.), an animal feed, a body of water or sewage (e.g., pond, lake, river, ocean, sewage channels, drinking water, etc.), a clinical specimen (e.g., blood, plasma, serum, sputum, tissue, urine, saliva, sample/fluid from the respiratory tract, etc.), soil, and cosmetic and pharmaceutical products (e.g., lotions, creams, ointments, solutions, medicines, eye and ear drops, etc.).

As used herein, the term “calibration curve” refers to a graphical presentation of the functional relationship between the expected value of the observed signal to the amount or concentration of analyte.

As used herein, the term “analyte standard” refers to materials containing a known concentration of an analyte. Analyte standards provide a reference to determine unknown concentrations or to calibrate analytical instruments.

As used herein, the term “elution solution” refers to a solution that desorbs (or elutes) the analyte from the column material by breaking the specific binding of the analyte with an affinity ligand. Determining the salt, pH and ionic conditions necessary for such functionality of elution solution is well known in the art.

As used herein, the term “wash solution” refers to a solution that removes unwanted substances from the sample loaded to the affinity column. Wash solution is applied in a washing step prior to the elution step, and its use may result in high yields and high concentrations of an analyte of interest eluted from the affinity column while effectively removing the unwanted substances in the matrix.

As used herein, the term “lateral flow diluent” refers to a solution that is compatible with a lateral flow test.

As used herein, the term “affinity ligand” refers to a substance (e.g. a functional group) that selectively captures (binds to) a target molecule from a mixture of molecules based on specific affinity between molecules e.g. an antigen and antibody binding.

As used herein, the term “aptamers” refers to a nucleic acid molecule that is capable of binding to a target molecule, such as a polypeptide (Tuerk and Gold, Science 249:505 (1990); Ellington and Szostak, Nature 346:818 (1990)).

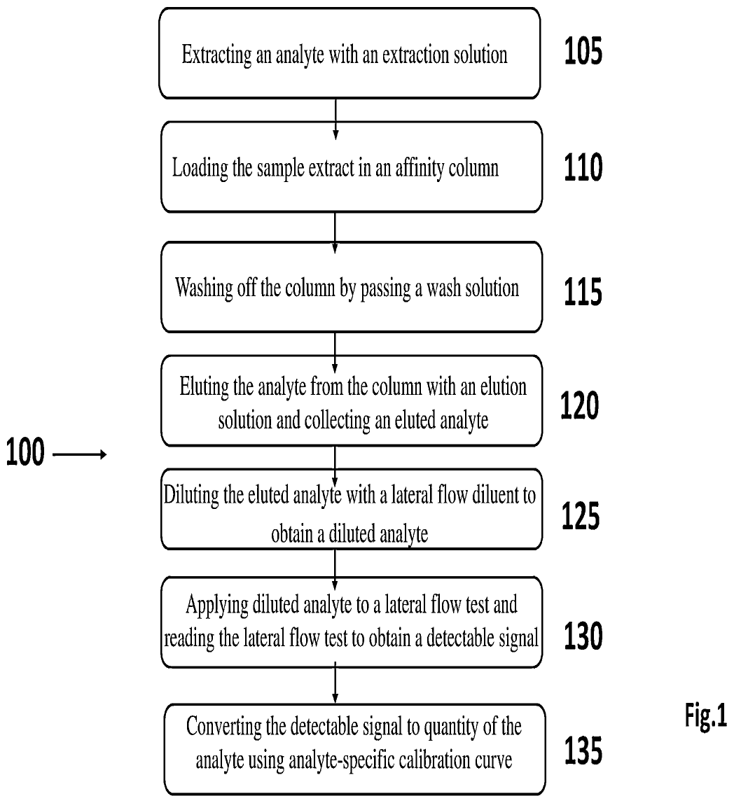

FIG. 1 illustrates a method 100 of detecting quantity of an analyte within a sample, including the steps of: Extracting an analyte 105 with an extraction solution to obtain a sample extract, wherein quantity of the analyte is unknown; Loading the sample extract in a column 110, wherein the column comprises an affinity resin; Washing off the column 115 by passing a wash solution through the column; Eluting the analyte from the column with an elution solution and collecting an eluted analyte 120, wherein volume of the elution solution is known; diluting the eluted analyte with a lateral flow diluent to obtain a diluted analyte 125, wherein volume of the lateral flow diluent is known; applying diluted analyte to a lateral flow test and reading the lateral flow test to obtain a detectable signal 130; converting the detectable signal from the lateral flow test to a numerical value related to the quantity of the analyte using an analyte-specific calibration curve, wherein the calibration curve correlates the detectable signals to quantity of the analyte 135.

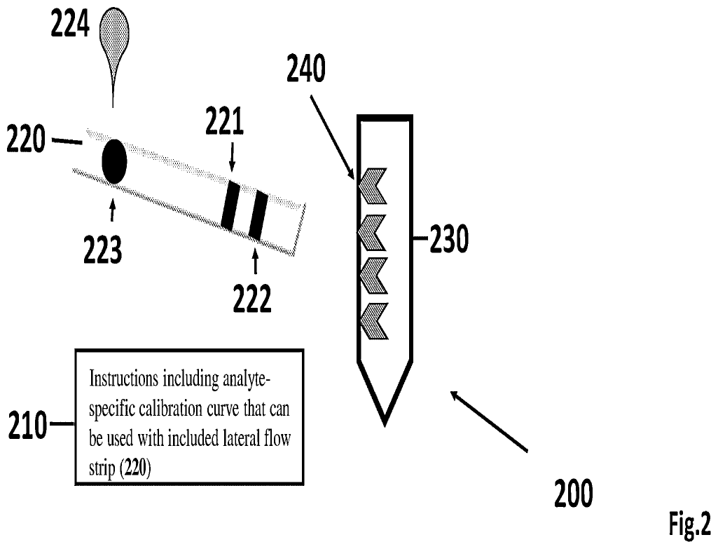

FIG. 2 illustrates a kit 200 for detecting the quantity of an analyte within a sample, wherein quantity of the analyte is unknown. Kit 200 includes a lateral flow test 220, including a strip for reacting with an analyte; and instructions 210 including analyte-specific calibration curve and how to use the calibration curve for quantification of an analyte using lateral flow test strip. The kit may also include a column 230 comprising an affinity resin coated with affinity ligands 240. Column 230 is an optional component of the kit. The lateral flow test strip can be placed contact with the liquid eluted analyte 224 which is diluted with suitable solvent (e.g. lateral flow diluent) to quantitatively determine the concentration of an analyte in the sample. The sample pad 223 is the first component of the lateral flow test strip that comes into contact with the eluted analyte 224. For example, the lateral flow test strip can draw the solution into the lateral flow device and to an indicator region. In some embodiments, the indicator region can be configured to display a control indicator line 222 and a test indicator line 221. The kit 200 includes the analyte-specific calibration curve included in the instructions 210 for the included flow test 220. The column 230, while being an optional kit component, is needed for use in sample preparation to enrich the analyte and to remove interfering matrix considerations to correlate with the included calibration curve. That is, while the included calibration curve can be used for any matrix the analyte is present in, the sample including the analyte needs to be cleaned using a sample preparation step to remove the matrix effects.

The calibration curve included in the instructions 210 can be provided as an algorithm which is then used to correlate a reading from the test strip into a quantity of analyte. The algorithm can be provided as software on a computer readable medium. In other embodiments, the algorithm can be provided in the kit as instructions and/or a link to download the algorithm from a particular server or network. In some embodiments, the algorithm is provided within a code, such as a QR code included within the instructions.

The calibration curve included in the instructions 210 is a universal calibration curve (i.e., one that is free from matrix effects) that can be used to determine the quantity of an analyte that is free from matrix effects.

### Affinity Chromatography

Affinity chromatography can be used in assays for purification and/or concentration of analytes. In some examples, purification and/or concentration occurs prior to examination by another technique. The present disclosure relates to use of affinity based chromatography (e.g. immunoaffinity) in sample preparation prior to lateral flow test.

Affinity chromatography (AC) columns offers superior specificity and enrichment. Affinity based chromatography may provide analytes to lateral flow test with substantially free of matrix effect.

Affinity chromatography is one of the most diverse and powerful chromatographic methods for purification of an analyte from complex samples. Some analytes that have been isolated by this process include proteins, glycoproteins, carbohydrates, lipids, bacteria, viral particles, drugs and environmental agents.

AC is based on highly specific interactions between two molecules, such as interactions between enzyme and substrate, receptor and ligand, or antibody and antigen. These interactions, which are typically reversible, are used for purification by placing one of the interacting molecules, referred to as affinity ligand, onto a solid matrix to create a stationary phase while the target molecule is in the mobile phase. The affinity columns can contain affinity ligands for single analytes or multiple analytes. Affinity ligands can be selected from analyte specific proteins, antibodies, aptamers, peptides, molecularly imprinted polymers (MIP), or synthetic small-molecular-weight compounds.

In one example, the sample is extracted with a suitable extraction solvent. In some embodiments, the suitable extraction solvent may be selected from various organic solvents, water, or mixture thereof. In one embodiment, the organic solvent can be selected from aliphatic solvents, aromatic solvents, carbonyl solvents, or a mixture thereof.

In one embodiment, the organic solvent can be selected from methanol, ethanol, acetonitrile, or a mixture thereof.

In another example, after samples are extracted, diluted and filtered, an affinity column can be loaded with a sample containing the analyte. The sample flow through the column under positive pressure applied on the top or by using a vacuum manifold. The sample also can flow through the column by gravity. Non-retained sample components are allowed to wash through the column. Washing removes any unwanted materials from affinity column while retaining materials that are bound to the resin, i.e., the targeted analytes. The analyte is then later eluted by disrupting the interaction between the antibody and the analyte with an elution solution. The collected sample, the eluate with the analyte, can be immediately analyzed or transferred to another device for analysis. Washing and elution can be performed the same way as sample loading.

The affinity column can generally be washed with water or buffer (e.g., a saline and/or phosphate based buffer) to remove the non-retained unwanted sample components.

Following washing the unwanted materials from the affinity column, the method includes eluting the analyte through the column with an elution solution to obtain the analyte which is substantially free of matrix effect. An elution solution disrupts the antibody-analyte interaction. Elution solutions include organic solvents (e.g., ethanol, methanol, acetonitrile, etc.). In some embodiments, the organic solvents can be diluted or concentrated to various degrees.

Enriching the analyte by washing away unwanted materials from the sample and concentrating the analyte on affinity ligands within the affinity column allows removal of matrix effects.

In some embodiments, the elution solution can be a buffer, and the buffer can be highly acidic or basic. In some embodiments, the buffers can have various salt content (i.e., ionic strength of the buffer can be set at a desired level or could vary over the elution). In some embodiments the buffers can include denaturants such as guanidine hydrochloride or urea, or the like. By flowing the elution solution through affinity column, the analyte can be released from the affinity resin. The elution solution can be selected based on the properties of the affinity resin and the analyte. Different strength elution solutions can be used. For example, for a strong interaction between the affinity resin and the analyte, a strong buffer may need to be used in order to overcome the interaction between the immunoaffinity resin and the analyte and to release the analyte from the immunoaffinity resin.

In some embodiments, the affinity column is an immunoaffinity resin. The immunoaffinity resin can be selected to retain a specific analyte or analyte(s). The immunoaffinity resin can include analyte specific antibodies, aptamers, or molecularly imprinted polymers (MIP). One example of an immunoaffinity resin includes beads sold under the name Sepharose® having a bead diameter that ranges from about 40 μm to about 200 μm (available from GE Healthcare, Chicago, Ill.). Other bead diameters may be used as well, including 100 microns. Immunoaffinity column (IAC) can be an IAC mini column and may contain antibodies for single analyte or multiple analytes.

In some examples, affinity column can be similar to columns sold in kits available from VICAM such as, for example, AflaOchra HPLC™, AOZ HPLC™, Myco6in1+® LC/MS/MS, AflaTest®, AflaB™, Afla M1™, AflaTest WB™, AflaTest WB SR™, OchraTest™, OchraTest WB™, FumoniTest™, FumoniTest WB™, ZearalaTest™, ZearalaTest WB™, DONtest HPLC™ DONtest WB™, DON-NIV™ WB, T-2test™, T-2/HT-2™ HPLC, Afla M1 FL⁺®, CitriTest®, and BPATest® (available from VICAM, Milford, Mass.).

The method and kit of present disclosure enables analysis of a single sample that contains one or more analytes. The affinity resins (e.g. immunoaffinity resins) can have the antibodies bound thereto. The immunoaffinity resin may contain multiple antibodies to purify multiple analytes at once. Accordingly, the method and the kit can use individual affinity columns to prepare a sample with a plurality of analytes.

The affinity resin can include multiple affinity ligands, and each affinity ligand can have specificity for an analyte. For example, the affinity resin can include a first affinity ligand with specificity for first analyte and a second affinity ligand with specificity for second analyte. The number of affinity ligands of the affinity resin can correspond to the number of analytes that are targeted.

The total amount of resin in an affinity column can vary according to a desired percent recovery of the analytes in the sample (e.g., 75%, 80%, 81%, 82%, 85%, 87%, 89%, 90%, 93%, or more) as well as a desired column flow rate (e.g., less than or about 3 mL/min, less than or about 1 drop/sec).

In addition to removing undesirable sample components, Affinity chromatography can concentrate analytes. The eluted fraction can be collected, dried down, and dissolved in a solvent more suitable for analysis. Drying and reconstituting can be done to change the solvent or concentrate the sample if necessary. For example, after collecting the eluted sample, the eluted sample can be dried or reconstituted to change a solvent of the method or concentrate the eluted sample. In some examples, a buffer exchange can be done by drying and reconstituting with a desired solvent.

### Analyte

In some embodiments, mycotoxins are the targeted analyte. Mycotoxins are toxic compounds elaborated by fungi. Some examples of mycotoxins include aflatoxin, ochratoxin, trichothecenes such as deoxynivalenol (DON), nivalenol, T2/HT2 toxin, patulin, zearalenone, citrinin, fumonisin or their analogs. The sample can contain a single mycotoxins or multiple mycotoxins.

Several compounds have been shown to belong to the aflatoxin group. Members of the aflatoxin group include B1, B2, G1, G2, M1, and M2. The aflatoxins have similar structures and form a group of highly oxygenated, naturally occurring heterocyclic compounds that fluoresce upon exposure to ultraviolet light. Members of the ochratoxin group include ochratoxin A, ochratoxin B, ochratoxin C, and ochratoxin TA and are structurally related derivatives of 3,4-dihydro-3-methylisocoumarin linked by an amide bond to L-beta phenylalanine at the 7-carboxy group. Trichothecenes, such as deoxynivalenol (DON) and related sesquiterpene alcohols, form another class of mycotoxins. The trichothecenes are a group of some 50 biologically active sesquiterpenes produced by various species of fungi. They are chemically characterized by a 12,13-epoxy-trichothec-9-ene ring system. Zearalenone, also known as RAL and F-2 mycotoxin, is a potent estrogenic metabolite produced by some Fusarium and Gibberella species. Members of the zearalenone group include zearalanone, α-zearalenol, β-zearalenol, α-zearalanol, β-zearalanol. Fumonisin is a group of mycotoxins derived from Fusarium and their Liseola section.

### Lateral Flow Test

After an eluted analyte (which is substantially free of matrix effect through sample preparation methods disclosed herein) is obtained, the eluted analyte is applied to lateral flow test.

In some embodiments, the eluted analyte is diluted with a suitable solvent (e.g. a lateral flow diluent) prior to lateral flow test. In one or more embodiments, the eluted analyte is diluted with a lateral flow diluent. In some embodiments, the lateral flow diluent can be similar to solutions available from VICAM such as, for example, AQUA premix solution, AQUA solution A, AQUA solution B (available from VICAM, Milford, Mass.).

In some embodiments, the lateral flow test can include a lateral flow device having a lateral flow test strip. The lateral flow test strip can be placed contact with the liquid eluted analyte which is diluted with suitable solvent, in the vial to quantitatively determine the concentration of an analyte in the sample. For example, the lateral flow test strip can draw the solution into the lateral flow device and to an indicator region. In some embodiments, the indicator region can be configured to display a control indicator line and a test indicator line.

In accordance with various embodiments, the control indicator line of the lateral flow device can be configured to appear at the conclusion of any successful test. In other words, the absence of the control indicator line can indicate that the test has failed. Test failure can occur, for example, due to improper preparation of the sample or probe material. In accordance with various embodiments, the degree to which the test indicator line appears at the conclusion of a successful test can correlate to the concentration of the analyte in solution. In some embodiments, a test indicator line that appears at the same intensity as the control indicator line can indicate that the analyte is not present in the sample. In other words, the test indicator line is absent when the concentration of the analyte is at or above a testing limit. Intensity values for the test indicator line between these extremes can be proportionate to the concentration value of the analyte in solution.

In some embodiments, the ratio of the light absorption value for the test line to the value for the control line (T/C ratio) can be used to quantitatively measure the response of the system. In other embodiments, only test line (T) or only control line (T) can be used to quantitatively measure the response of the system.

In some embodiments, a lateral flow reader such as the Vertu (commercially available from VICAM, Milford, Mass.) can be used to analyze the indicator on the lateral flow device. The lateral flow reader can illuminate the lateral flow device and measure the absorption or extinction of light caused by the indicator. The absorption or extinction can be indicative of a concentration of an analyte in solution.

In some embodiments, the lateral flow test may comprise more than one test line for quantifying more than one analytes simultaneously.

### Methods/Techniques/Results

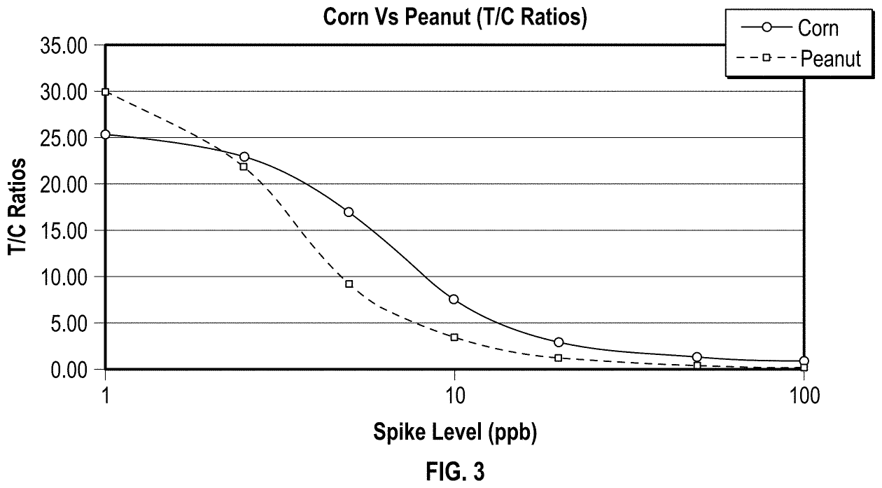

In order to understand matrix effect on calibration curves, lateral flow test was performed on corn and peanut samples. Aflatoxin was chosen as an analyte of interest. In order to obtain a specific calibration curve for each sample, both corn and peanut samples were extracted with methanol (80% v/v), and then spiked with different concentrations of Aflatoxin. Table 1 show spike levels (ppb) for corn and peanut samples. Prior to lateral flow tests, samples are diluted with Aqua Premix Solution (commercially available from VICAM, A Waters Business, Milford, Mass.). Methanol/Aqua Premix is applied to lateral flow test as a control. Based on Test line/Control line (T/C) ratios obtained from the lateral flow reader, Vertu (commercially available from VICAM, Milford, Mass.), calibration curves shown in FIG. 3 were obtained.

Calibration curves obtained for spiked corn extract (i.e., corn matrix) and spiked peanut extract (i.e., peanut matrix) do not superimpose at the same spike levels of Aflatoxin. In other words, even though Aflatoxin is present at the same level in both corn extract and peanut extract, lateral flow test provides different T/C values for each sample.

TABLE 1

| Lot No: FG035-014 | T/C Ratios | |
|---|---|---|
| Spike Level (PPb) | Corn | Peanut |
| 0 | 33.63 | 37.64 |
| 1 | 25.38 | 29.94 |
| 2.5 | 23.08 | 22.02 |
| 5 | 17.17 | 9.28 |
| 10 | 7.65 | 3.45 |
| 20 | 3.00 | 1.37 |
| 50 | 1.03 | 0.38 |
| 100 | 0.53 | 0.19 |

In order to obtain Aflatoxin substantially free from interfering compounds of matrix, corn and peanut samples are extracted with 80% v/v methanol, diluted, filtered and loaded into an immunoaffinity column packed with resins coupled with antibodies specific to Aflatoxin. The Aflatoxin is captured by antibodies, and the impurities are washed off by passing a wash solution through the column. After washing, the Aflatoxin is eluted with elution solution to break the interactions between antibodies and Aflatoxin. The eluate is collected and diluted with Aqua Premix Solution (commercially available from VICAM, A Waters Business, Milford, Mass.). Prior to applying the analyte to lateral flow test, it was spiked with 10 ppb Aflatoxin.

TABLE 2

| | T/C Ratios | |
|---|---|---|
| Spike Level | Corn | Peanut |
| (PPb) | (with AC cleanp) | (with AC clean-up) |
| 10 | 10.28 | 10.33 |
| | 10.39 | 10.03 |
| | 9.71 | 10.44 |
| | Corn | Peanut |
| | (without AC clean-up) | (without AC clean-up) |
| | 6.447 | 3.091 |
| | 6.926 | 4.032 |
| | 6.017 | 3.621 |

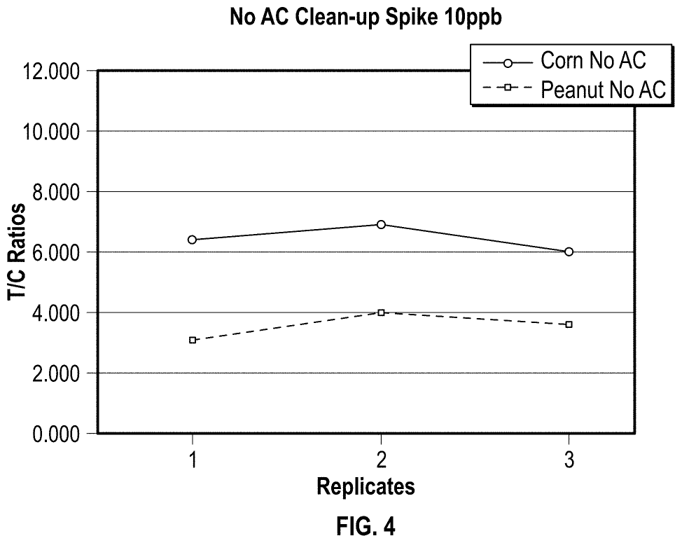

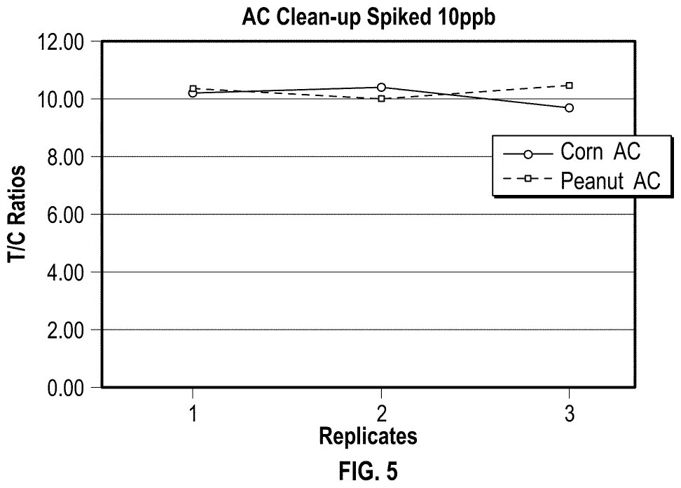

Table 2 shows (T/C) ratios for peanut and corn samples with and without affinity column sample preparation step. Three replicates were analyzed for each sample. FIG. 4 and FIG. 5 shows T/C ratios of these three replicates for each sample with and without affinity chromatography sample preparation step.

Results clearly show that both peanut and corn samples have different matrices causing variations in T/C ratios at the same spike level of aflatoxin. This means that even if same amount of analyte is present in two samples, T/C ratios can be different due to matrix effect. On the other hand, when the matrix is removed by affinity chromatography sample preparation step (FIG. 5), T/C ratios of both samples superimpose at the same spike level of Aflatoxin.

These findings pave the way for using one universal analyte-specific calibration curve for all samples regardless of their matrices if the sample is purified through an affinity column. Therefore, the methods and kits of the present disclosure removes the need for generating and employing numerous calibrations for each sample to cope with various matrix effect.

The present technology which uses affinity column chromatography as a sample preparation technique to clean matrix effects from a sample allows generation of calibration curves per analyte regardless of the sample where the analyte is present in. In some embodiments, provided herein lateral flow test strips for quantitative analysis of specific analytes and calibration curves specific to said analyte. To illustrate, ordinary skill in the art can use the analyte-specific calibration curve disclosed in various embodiments of the present disclosure to quantify aflatoxin in various samples such as food samples e.g. dried fruits, tree nuts, grains etc. or in various lots of samples such as different lots of feed samples e.g. different corn samples.

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements, device components or method steps, those elements, components or steps may be replaced with a single element, component, or step. Likewise, a single element, component, or step may be replaced with a plurality of elements, components, or steps that serve the same purpose. Moreover, while exemplary embodiments have been shown and described with references to particular embodiments thereof, those of ordinary skill in the art will understand that various substitutions and alterations in form and detail may be made therein without departing from the scope of the invention. Further still, other embodiments, functions, and advantages are also within the scope of the disclosure.

1. (canceled)

2. A method of detecting quantity of an analyte within a sample, wherein quantity of the analyte is unknown; comprising:
a) extracting the analyte with an extraction solution to obtain a sample extract;
b) loading the sample extract in a column, wherein the column comprises an affinity resin;
c) washing off the column by passing a wash solution through the column;
d) eluting the analyte from the column with an elution solution;
e) collecting an eluted analyte;
f) diluting the eluted analyte with a lateral flow diluent to obtain a diluted analyte;
g) applying diluted analyte to a lateral flow test;
h) reading the lateral flow test to obtain a detectable signal; and
i) converting the detectable signal from the lateral flow test to a numerical value related to the quantity of the analyte using an analyte-specific calibration curve of claim 1, wherein the calibration curve correlates the detectable signals to quantity of the analyte.

3. The method of claim 2, wherein one analyte-specific calibration curve is capable of detecting quantity of the same analyte within different samples.

4. (canceled)

5. The method of claim 2, wherein the affinity resin comprises affinity ligands selected from analyte specific proteins, antibodies, aptamers, peptides, molecularly imprinted polymers (MIP), or synthetic small-molecular-weight compounds.

6. The method of claim 2, wherein the affinity resin comprises an immunoaffinity resin including analyte specific antibodies.

7. The method of claim 2, wherein the sample contains a single analyte or multiple analytes.

8. The method of claim 5, wherein the affinity resin contains multiple affinity ligands to purify multiple analytes at once.

9. The method of claim 6, wherein the immunoaffinity resin contains multiple analyte specific antibodies to purify multiple analytes at once.

10. The method of claim 2, wherein the detectable signal is test to control (T/C) ratios.

11. The method of claim 2, wherein the extraction solution comprises organic solvent, water, or a mixture thereof.

12. The method of claim 11 wherein the organic solvent is selected from methanol, ethanol, acetonitrile, or a mixture thereof.

13. The method of claim 2, wherein the sample is selected from an agricultural sample, an environmental sample, or a biological sample.

14. The method of claim 2, wherein the sample is animal feed or subsequent quantification thereof.

15. The method of claim 2, the analyte is selected from mycotoxins, allergens, antibiotics.

16. The method of claim 15, wherein the mycotoxins are selected from the group of aflatoxin, ochratoxin, deoxynivalenol, nivalenol, T2/HT2 toxin, patulin, zearalenone, citrinin, fumonisin or their analogs.

17. The method of claim 16, wherein the aflatoxin is selected from the group of aflatoxin B1, aflatoxin B2, aflatoxin G1, aflatoxin G2, aflatoxin M1, or aflatoxin M2.

18. A kit for detecting quantity of an analyte within a sample, wherein quantity of the analyte is unknown, the kit comprising:
a) optionally, a column comprising an affinity resin;
b) a lateral flow test including a strip for reacting with the analyte;
c) an algorithm corresponding to an analyte-specific calibration curve, wherein the analyte-specific calibration curve does not change for use in detecting the analyte in different matrix materials; and
d) instructions for reading the lateral flow test strip and determining quantity of an analyte based on the algorithm of step c).

19. The kit of claim 18 further comprising sample preparation instructions for removing matrix effects from the sample.

20. The kit of claim 18, wherein the sample preparation instructions include use of the optional column.

21. The kit of claim 18, wherein the sample is a complex mixture.

22. The kit of claim 21, wherein the complex mixture is a food source or food product.

23. (canceled)

24. The kit of claim 18, wherein the analyte is selected from mycotoxins, allergens, antibiotics.

25. (canceled)

26. (canceled)
